(19) United States
(12) Patent Application Publication
(10) Pub. No.: US 2007/0026047 A1
(43) Pub. Date: Feb. 1, 2007

(54) Thermal effect on crystalinity for drug delivery devices

(76) Inventors: Li-Chun Tsou, Cockeysville, MA (US); Sharon Myers, Webster, NY (US); Frank Price JR., Webster, NY (US); David Heiler, Avon, NY (US); James Bonafini, Pittsford, NY (US); Susan Spooner, Cary, NC (US)

Correspondence Address: Bausch & Lomb Incorporated, One Bausch & Lomb Place, Rochester, NY, 14604-2701, US

(21) Appl. No.: 11/472,202

(22) Filed: Jun. 21, 2006

Related U.S. Application Data
(60) Provisional application No. 60/692,664, filed on Jun. 21, 2005.

(51) Int. Cl. A61F 2/00

(52) U.S. Cl. 424/427

(57) ABSTRACT

A drug delivery device for placement in the eye includes a drug core comprising a pharmaceutically active agent, and a holder that holds the drug core. The holder is made of a material impermeable to passage of the active agent and includes an opening for passage of the pharmaceutically agent therethrough to eye tissue. The device includes a layer of material permeable to passage of the active agent. The material permeable to passage of the active agent has a degree of crystallinity selected to influence the release kinetics.

### CROSS REFERENCE

This application claims the benefit of Provisional Patent Application No. 60/692,664 filed Jun. 21, 2005 and is incorporated herein by reference.

### FIELD OF THE INVENTION

This invention relates generally to drug delivery devices utilizing the crystallinity of a polymeric diffusion barrier to control drug release characteristics. In one embodiment the device is placed or implanted in the eye to release a pharmaceutically active agent to the eye with near zero-order kinetics. The device includes a drug core and a holder for the drug core, wherein the holder is made of a material impermeable to passage of the active agent and includes at least one opening for passage of the pharmaceutically agent therethrough to eye tissue. Particularly, this invention provides improved methods of making such devices by tailoring the crystallinity of the polymeric diffusion barrier to the active agent and the desired release characteristics.

### BACKGROUND OF THE INVENTION

Various materials have been developed for the controlled release of drugs. For example, PCT/US02/18355 (Orgill et al.) discloses biocompatible polymers which can incorporate drug for controlled release. Orgill et al also disclose cross-linked gels of natural biomolecules. U.S. patent application Ser. No. 09/692,664 (Lee et al.) discloses an anticancer composition comprising a mixture of an anticancer agent and a calcium phosphate paste. This reference also discloses that control of the calcium phosphate cement degree of crystallinity and crystal size may be used to affect the overall vehicle absorption rate. Thus, there is still a need in the art for additional methods of controlling the release rate from drug delivery devices.

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye and treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); U.S. patent application Ser. No. 10/610,063 (Drug Delivery Device, filed Jun. 30, 2003) (Mosack) and U.S. patent application Ser. No. 11/006,915 (Drug Delivery Device, filed Dec. 8, 2004) (Renner, et al.).

Many of these devices include an inner drug core including a pharmaceutically active agent, and some type of holder for the drug core made of an impermeable material such as silicone or other hydrophobic materials. The holder includes one or more openings for passage of the pharmaceutically active agent through the impermeable material to eye tissue. Many of these devices include at least one layer of material permeable to the active agent, such as polyvinyl alcohol.

Various prior methods of making these types of devices involve the step of heat curing one of the materials from which the device is fabricated, such as the layer of permeable material, after insertion of the drug core in the device. The present disclosure is based on the discovery that crystallinity is sensitive to process conditions. Different degrees of crystallinity provide a full range of options that suit specific requirements in designing biomedical devices. More specifically, PVA hydrogels with unique semi-crystalline structures allow for the customization of their physical and chemical properties to fulfill design requirements demanded by the pharmaceutical and medical industry.

### BRIEF DESCRIPTION OF THE DRAWINGS

FIG. 1 is a perspective view of a first embodiment of a drug delivery device of this invention;

FIG. 2 is a cross-sectional view of the device of FIG. 1;

FIG. 3 is a cross-sectional view of the device of FIGS. 1 and 2 during assembly;

FIG. 4 is a cross-sectional view of a second embodiment of a drug delivery device;

FIG. 5 is a graphical representation of the release profile of a device made according to the invention herein as compared to a prior art device;

FIG. 6 is a graphical representation of the long term release profile of a device made according to the invention herein;

FIGS. 7A and B are graphical representations of the impurity content of PVA raw materials via Thermal Gravimetric Analysis (TGA);

FIG. 8 is a graphical representation of the molecular weight effect on viscosity via rheometer;

FIG. 9 is a graphical representation of the process effect on water content of dry PVAA film via TGA;

FIG. 10 is a graphical representation of the thermal treatment effect on diffusion rate through PVA films;

FIG. 11 is a graphical representation of DSC showing a water peak at 110° C. in pre-cured film;

FIG. 12 is a graphical representation of DSC showing a shoulder peak at 120° C. in post-cure films;

FIG. 13 is a graphical representation of DSC showing higher crystallinity in post-cure hydrogels;

FIG. 14 is a graphical representation of DSC showing smaller enthalpy was in pre-cure hydrogels;

FIG. 15 is a graphical representation of the stress-strain curves of PVA hydrogels (MW=55,000);

FIG. 16 is a graphical representation of a DSC thermograph of hydrated PVA films;

FIG. 17 is a graphical representation of tensile properties of PVA hydrogels (MW=77,000);

FIG. 18 is a graphical representation of the crystallinity effect on elastic modulus;

FIG. 19 is a graphical representation of multi cycle recovery of PVA hydrogels thermally treated for 7 hours at 135° C.;

FIG. 20 is a graphical representation of the crystallinity effect on multicycle recovery;

FIG. 21 is a graphical representation of multi cycle recovery of PVA hydrogels thermally treated for 7 hours at 150° C.;

FIG. 22 is a graphical representation of multi cycle recovery of PVA hydrogels (MW=77,000);

FIG. 23 is a graphical representation of multi cycle recovery of PVA hydrogels thermally treated for 7 hours at 135° C.;

FIG. 24 is a graphical representation of the crystallinity effect on modulus & recovery.

### DETAILED DESCRIPTION OF PREFERRED EMBODIMENTS

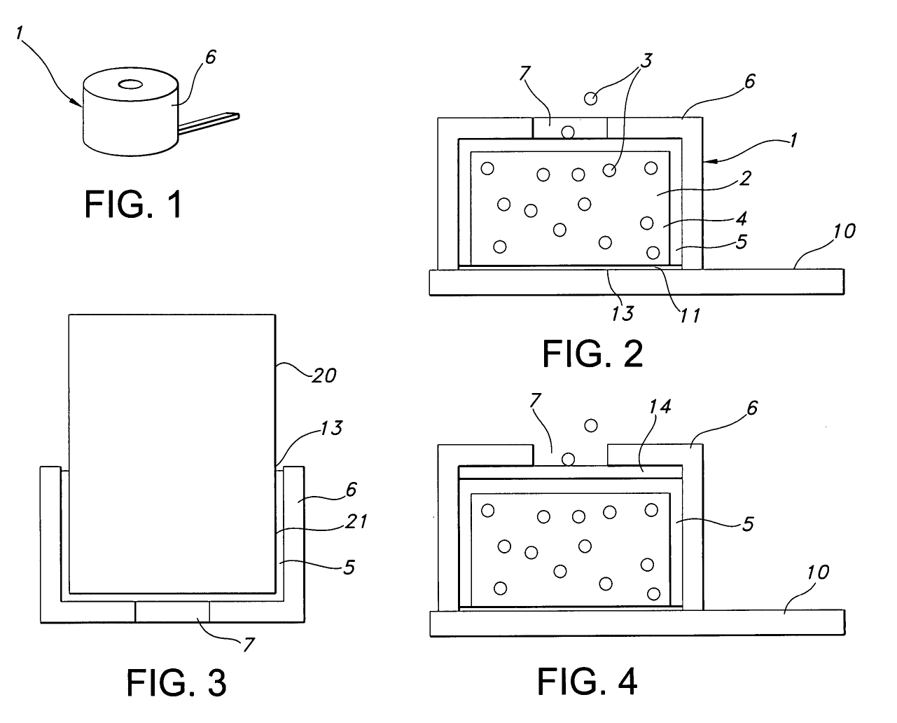

FIGS. 1 and 2 illustrate a first embodiment of a device of this invention. Device 1 is a sustained release drug delivery device for implanting in the eye. Device 1 includes inner drug core 2 including a pharmaceutically active agent 3.

This pharmaceutically active agent may include any compound, composition of matter, or mixture thereof that can be delivered from the device to produce a beneficial and useful result to the eye, especially an agent effective in obtaining a desired local or systemic physiological or pharmacological effect. Examples include: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen; nutrients and vitamins; enzyme inhibitors; antioxidants; anticataract drugs; aldose reductase inhibitors; cytoprotectants; cytokines, cytokine inhibitors, and cytokin protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, loteprednol etabonate, medrysone, methylprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; svmpathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

As shown in the illustrated embodiment, active agent 3 may be mixed with a matrix material 4. Preferably, matrix material 4 is a polymeric material that is compatible with body fluids and the eye. Additionally, matrix material should be permeable to passage of the active agent 3 therethrough, particularly when the device is exposed to body fluids. For the illustrated embodiment, the matrix material is PVA. Also, in this embodiment, inner drug core 2 may be coated with a coating 5 of additional matrix material which may be the same or different from material 4 mixed with the active agent. For the illustrated embodiment, the coating 5 employed is also PVA.

Device 1 includes a holder 6 for the inner drug core 2. Holder 6 is made of a material that is impermeable to passage of the active agent 3 therethrough. Since holder 6 is made of the impermeable material, at least one passageway 7 is formed in holder 6 to permit active agent 3 to pass therethrough and contact eye tissue. In other words, active agent passes through any permeable matrix material 4 and permeable coating 5, and exits the device through passageway 7. For the illustrated embodiment, the holder is made of silicone, especially polydimethylsiloxane (PDMS) material.

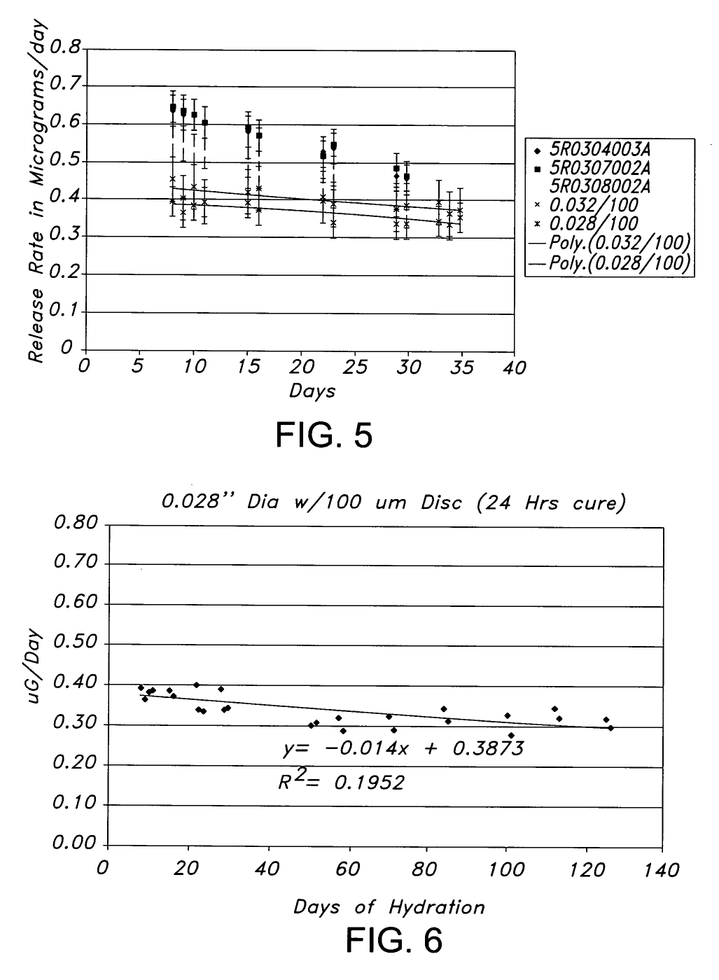

A prior method of making a device of the type shown in FIGS. 1 and 2 includes the following procedures. A cylindrical cup of silicone is separately formed, for example by molding, having a size generally corresponding to the drug core tablet and a shape as generally shown in FIG. 2. This silicone holder is then extracted with a solvent such as isopropanol. Openings 7 are placed in silicone, for example, by boring or with the laser. A drop of liquid PVA is placed into the holder through the open end 13 of the holder, this open end best seen in FIG. 3. Then, the inner drug core tablet is placed into the silicone holder through the same open end 13 and pressed into the cylindrical holder. As a result, the pressing of the tablet causes the liquid PVA to fill the space between the tablet inner core and the silicone holder, thus forming permeable layer 5 shown in FIGS. 1 and 2. For the illustrated embodiment, a layer of adhesive 11 is applied to the open end 13 of the holder to fully enclose the inner drug core tablet at this end. Tab 10 is inserted at this end of the device. The liquid PVA and adhesive are cured by heating the assembly. FIG. 5 shows the improved release characteristics obtained through use of a device according to the invention herein as compared to a device prepared as is described in U.S. Pat. No. 6,217,895. As shown in FIG. 5, the device of the invention herein provides zero order or near-zero order release profile without an initial spike of drug released. FIG. 6 shows that this release profile can be maintained for at least 120 days.

As mentioned, this invention recognized that crystallinity is sensitive to process conditions. Different degrees of crystallinity provide a full range of options that suit specific requirements in designing biomedical devices, e.g., release profiles. More specifically, PVA hydrogels with unique semi-crystalline structures allowed us to customize their physical and chemical properties to fulfill the design requirements that were demanded by the pharmaceutical and medical industry.

A first embodiment of this invention is illustrated in FIG. 4. In this embodiment, the device further includes a disc 14 made of permeable material covering passageway 7 between the holder 6 and layer 5. For the illustrated embodiment, disc 14 may be preformed from PVA with a controlled degree of crystallinity. In assembling this embodiment, disc 14 is placed in holder 6 prior to adding the liquid curable material forming layer 5. Then, pin 20 is used to displace the liquid, as in the previous embodiment. A potential advantage of this embodiment is that the thickness of the permeable materials at passageway 7 can be controlled better, thereby providing more consistent release of active through the permeable materials into passageway 7.

Other semicrystalline polymeric materials suitable for biomedical application would include polyethylene (PE), polypropylene (PP), polyethylene terephthalate (PET), polytetrafluoroethylene (PTFE), polyamide (Nylon), and polycaprolactone (PCL). For examples, PE is used for catheter and orthopedic implants. PP can be used for blood oxygenator membrane and artificial vascular grafts. PET can be used in implantable suture and heart valve. PTFE can be used in catheter and artificial vascular grafts. Nylon can be used in catheters and sutures. PCL can be used for implants in hormone replacement therapy, glucose monitor/insulin pump, and anti-malarial sustained-release formulation.

According to a second embodiment, the invention utilizes the existence of crystalline phase to achieve mechanical integrity of, for example, PVA hydrogels and desirable release kinetics. Other semi-crystalline polymeric materials would include polyethylene and polypropylene. Such semi-crystalline polymers demonstrate a distinctive glass transition temperature and melting transition character as determined by differential scanning calorimetry. Differential Scanning Calorimetry (DSC) was chosen to characterize this intricate physical network by the formation of crystalline structure in PVA based films and hydrogels. This crystalline structure in PVA plays a role in enhancing mechanical strength and barrier efficiency. Different processes led to different degrees of crystallinity, which defined the physical-chemical properties of PVA for bio-medical applications. Crystallinity determination by using DSC provided a reliable means to monitor the process and to control the quality of product performance.

Impurity levels can affect the subsequent product performance for use in forming the permeable disc. A thermo-gravimetric analyzer (TGA) was used to characterize PVA raw materials as received and those purified through special request from vendors. Two types of PVA with M.W=55,000 and with M.W.=77,000 were purchased to prepare different weight ratios of PVA solution for film casting. Both weights of PVA can be mixed with, for example, triple-distilled purified water. Solution preparation can be vital since an aqueous PVA system may have a narrow process window. For instance, the PVA solution can be heated to 95° C. for 30 minutes to form a homogeneous solution. The chilled solution can be poured, for example, onto a glass plate to cast the film prior to a pre-heat treatment process. The pre-heat treatment film casting step generated clear and pliable films, whereas post-heat treatment processes promoted rigid and tough semi-crystalline films.

For a third embodiment, the active agent may be provided in the form of a micronized powder, and then mixed with an aqueous solution of the matrix material, in this case PVA, whereby the active agent and PVA agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 2.

In addition to the materials described above, a wide variety of materials may be used to construct the devices of the present invention. The only requirements are that they are inert, non-immunogenic and of the desired permeability. Materials that may be suitable for fabricating the device include naturally occurring or synthetic materials that are biologically compatible with body fluids and body tissues. Crystalline polymers can be tailored to high mechanical strength. They can also be tailored into biodegradable forms that can be flexible. In a fourth embodiment, semicrystalline polymers can be can be formed such that the drug delivery carrier is biodegradable. This embodiment may be useful as an outer coating for beads containing API (growth hormone/steroid) to treat a medical condition.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

The illustrated embodiment includes a tab 10 which may be made of a wide variety of materials, including those mentioned above for the matrix material and/or the holder. Tab 10 may be provided in order to attach the device to a desired location in the eye, for example, by suturing. For the illustrated embodiment, tab 10 is made of PVA and is adhered to the inner drug core 2 with adhesive 11. Adhesive 11 may be a curable silicone adhesive, a curable PVA solution, or the like. If it is not necessary to suture the device in the eye, element 10 may have a smaller size such that it does not extend substantially beyond holder 6.

According to preferred embodiments, the holder is extracted to remove residual materials therefrom. For example, in the case of silicone, the holder may include lower molecular weight materials such as unreacted monomeric material and oligomers. It is believed that the presence of such residual materials may also deleteriously affect adherence of the holder surfaces. The holder may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

If desired, the holder may be plasma treated, following extraction, in order to increase the wettability of the holder and improve adherence of the drug core and/or the tab to the holder. Such plasma treatment employs an oxidation plasma in an atmosphere composed of an oxidizing media such as oxygen or nitrogen containing compounds: ammonia, an aminoalkane, air, water, peroxide, oxygen gas, methanol, acetone, alkylamines, and the like, or appropriate mixtures thereof including inert gases such as argon. Examples of mixed media include oxygen/argon or hydrogen/methanol. Typically, the plasma treatment is conducted in a closed chamber at an electric discharge frequency of 13.56 Mhz, preferably between about 20 to 500 watts at a pressure of about 0.1 to 1.0 torr, preferably for about 10 seconds to about 10 minutes or more, more preferably about 1 to 10 minutes.

The device may be sterilized and packaged. For example, the device may be sterilized by irradiation with gamma radiation.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease state, type of mammalian organism, location of administration, and agents or agent administered are among the factors which would effect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

### EXAMPLES

Thermal Analysis (TGA, Rheometer and DSC)

Impurity evaluation was conducted in TGA, DuPont 951. Molecular weight effect and solute concentration on solution viscosity were evaluated by using a parallel plate Rheometer (AR1000). Crystallinity characterization of PVA hydrogels was measured via modulated differential scanning calorimetry (MDSC 2910). Melting temperature and endothermic enthalpy were analyzed. The degree of crystallinity was calculated by using the formula of % crystallinity=ΔH/ΔH₀, where ΔH, the area under the melting endotherm, can be measured, and ΔH₀, the heat of fusion on 100% crystalline PVA, can be found in the literature.

Tensile Properties (Elastic Modulus, Cyclic Recovery and Stress Relaxation)

Mechanical property and stress-strain curves gave excellent indication of polymer nature. Instron tensile tester (MTS 1/G) coupled with a hydration chamber were used to evaluate elastic modulus, elongation and tensile strength of PVA hydrogels. ASTM D-882 was used as a benchmark for test procedure. At least five specimens per process condition were tested and compared. The thickness of the specimen was measured by Rehder gauge, model E.T.-1., to the accuracy of ±0.5 μm. In general, the film thickness was controlled between 100 μm-150 μm.

Diffusion Rate

The diffusion rate was vital for the applications of poly(vinyl alcohol) hydrogels and films as permeation membranes. For example, the exchange of hemoglobin through PVA is known to be highly dependent on the degree of crystallinity and the mesh size of the crystallites in the barriers. See A. K. Bajpai, S. Bhanu, “In vitro release modulation of hemoglobin from a ternary polymeric delivery vehicle”, J. Appl. Polym. Sci., 2002, 85, 1, 104-113. To evaluate the process condition effect on the diffusion rate, UV-VIS spectroscopy was selected as a means to quantify the diffusion rate over time across the hydrogel membranes made of poly(vinyl alcohol). A diffusion-cell apparatus was constructed by using two cells and a membrane with a fixed thickness. One side of cell was filled with saturated API (active pharmaceutical ingredient) solution and the other side was filled with blank buffered salient. The measurement was taken at the initiation time zero and subsequently every 24 hours. The absorption was calculated by using Beer's law:
ε=abc
where ε represents the absorbance, a was the absorption coefficient, b is the width of cuvette and c was the concentration of the solution. Thermal Effect on diffusion rates was investigated.
Discussion

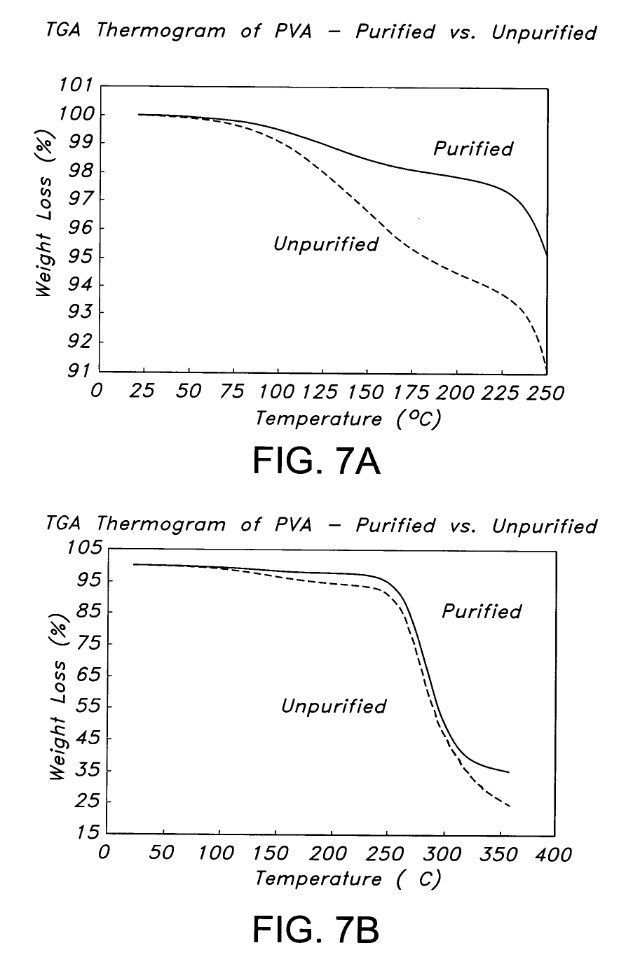

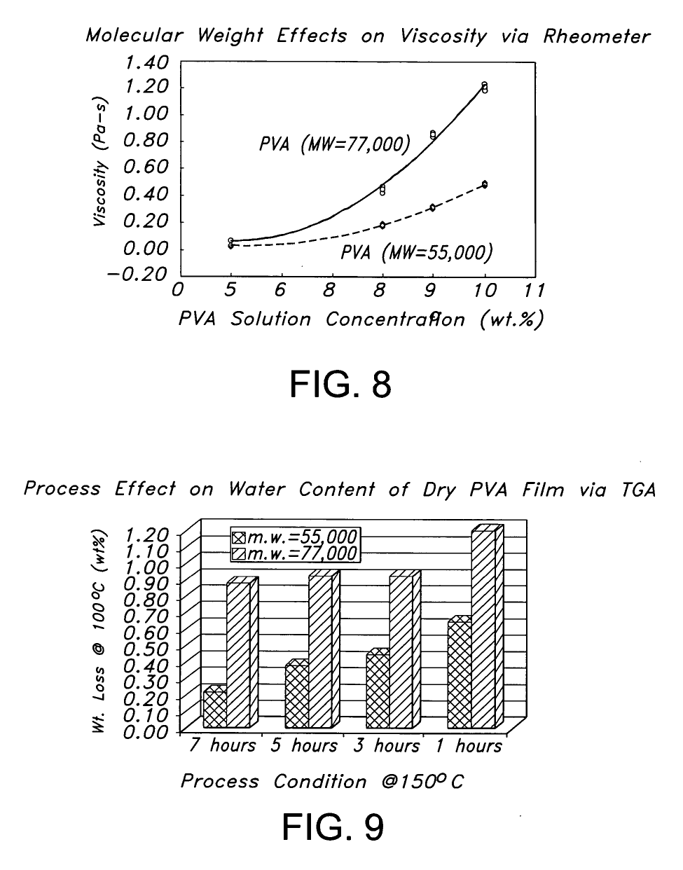

Impurity concerns were addressed by the TGA results. As shown in FIGS. 7A and B, we observed that purified PVA exhibited 1.5% volatile organic compounds whereas unpurified PVA exhibited about 4.99% impurity below 150° C. Higher viscosity was associated with higher concentration of PVA solution by using parallel plate Rheometer (FIG. 8). Solutions made of higher molecular weight of PVA exhibited higher viscosity. However, it was interesting to observe that lower crystallinity was detected in the hydrated PVA films made of high molecular weight of PVA via DSC. An increased water content further agreed with the DSC results showing that hydrogels made of higher molecular weight PVA seemed to have less crystallinity. It suggested that higher chain entanglement typically observed in higher molecular weight PVA may impede the crystalline formation during thermal treatment process.

Prolonged heat treatment reduced the water uptake at all groups regardless of their original molecular weight (FIG. 9). This suggests that higher temperature and longer thermal treatment time increased the molecular order. Consequently, the ratio of amorphous phase decreased based on the assumption that water uptake generally occurred in the amorphous region.

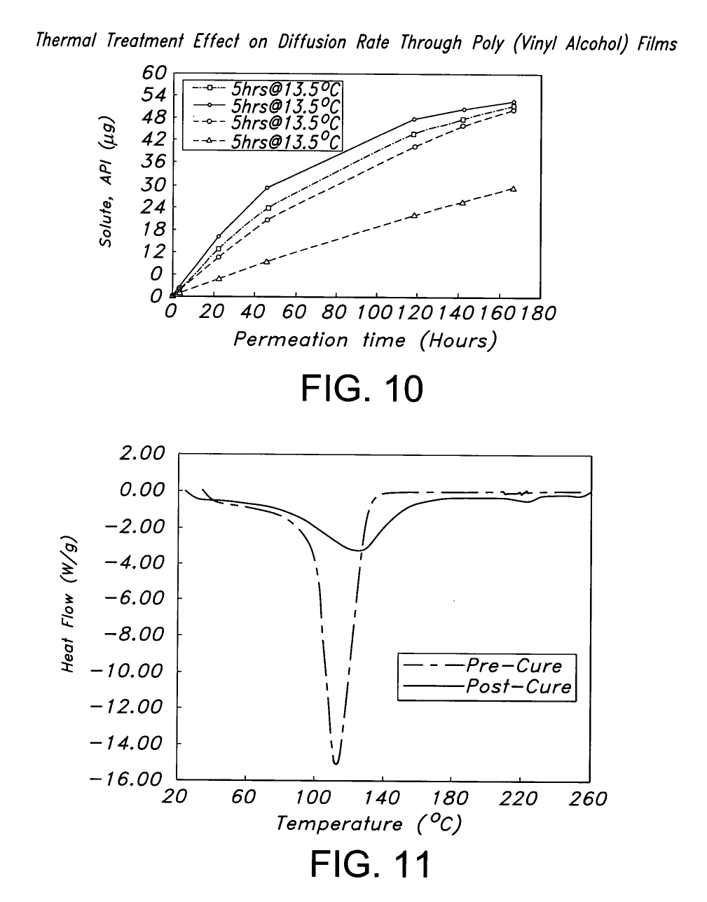

Diffusion rate was significantly correlated with the process condition as well (FIG. 10). Different process conditions were carried through the UV-VIS spectroscopy evaluation. The results showed that prolonged heat treatment reduced the diffusion rate in proportion to the heat treatment time and temperature (Table 1).

TABLE 1

| | | ΔH_{f} | ΔH_{f} | |
|---|---|---|---|---|
| | H₂O | (J/g) | (J/g) | Modulus |
| PVA Cure Condition | (%) | Wet | Dry | (g/mm²) |
| 3 hrs@135° C. | 44.99 | 23.8 | 66.59 | 584 |
| 5 hrs@135° C. | 42.68 | 27.7 | 66.60 | 803 |
| 3 hrs@150° C. | 39.68 | 33.4 | 63.87 | 1098 |
| 5 hrs@150° C. | 29.73 | 40.5 | 64.09 | 1643 |

It suggested that prolonged heat treatment promoted the formation of crystalline regions. Thus, lower diffusion rate was observed in those membranes exhibiting higher degree of crystallinity, whereas a greater diffusion rate was observed in the membranes exhibiting higher ratio of amorphous phase. In other words, the consistency of higher water content and greater diffusion rate observed in the Poly(vinyl alcohol) hydrogels subjected to shorter thermal treatment process were basically linked with relatively higher ratio of amorphous phase and lower degree of crystallinity. Swelling behavior of Poly(vinyl alcohol) was dictated by the percentage of amorphous phase, and the counter ratio of less immobile phases, such as crystallinity and cross-links. Some evidence indicates that the thermal processes promoted both crystallinity and the cross-links depending on the temperature level.

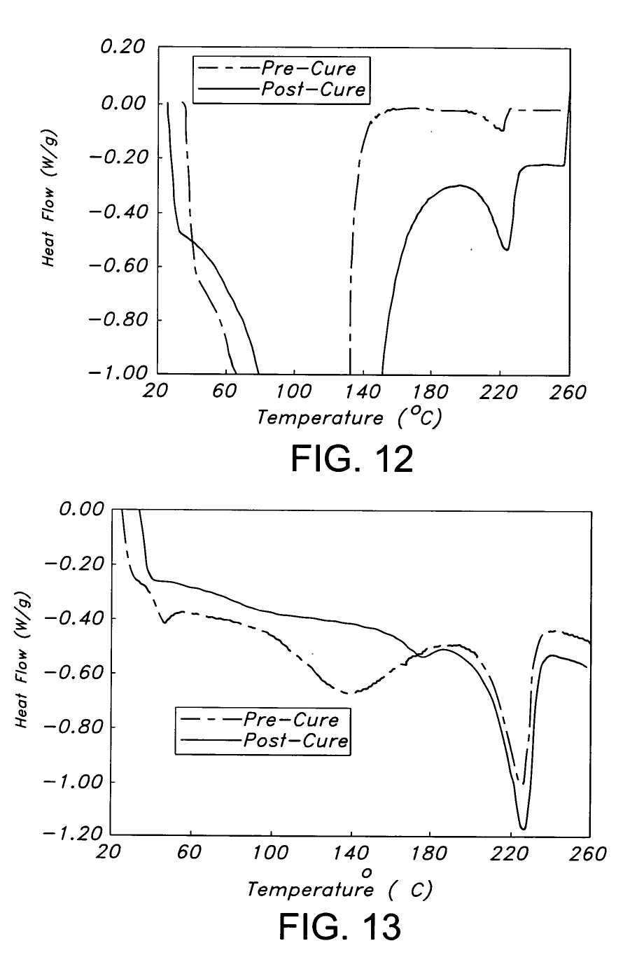

A typical DSC thermogram of PVA comprises both T_{g} and Tₘ. By comparing the shapes and areas under curves of DSC thermograms, we observed that PVA films thermally treated at higher temperature exhibited higher transition temperature and a larger area under curve. Pre-heat treatment film showed a narrower and distinctive water plastized peak at 110° C., whereas post-cure film exhibited a broader melt peak at 120° C. Both hydrogels had a prominent melting peak around 225° C. (FIGS. 11 and 12). However, the melting peak observed in pre-heat treatment film was trivial compared to the one in post-heat treatment film. Higher crystallinity was calculated from post-heat treatment film compared with lower crystallinity obtained from pre-heat treatment film.

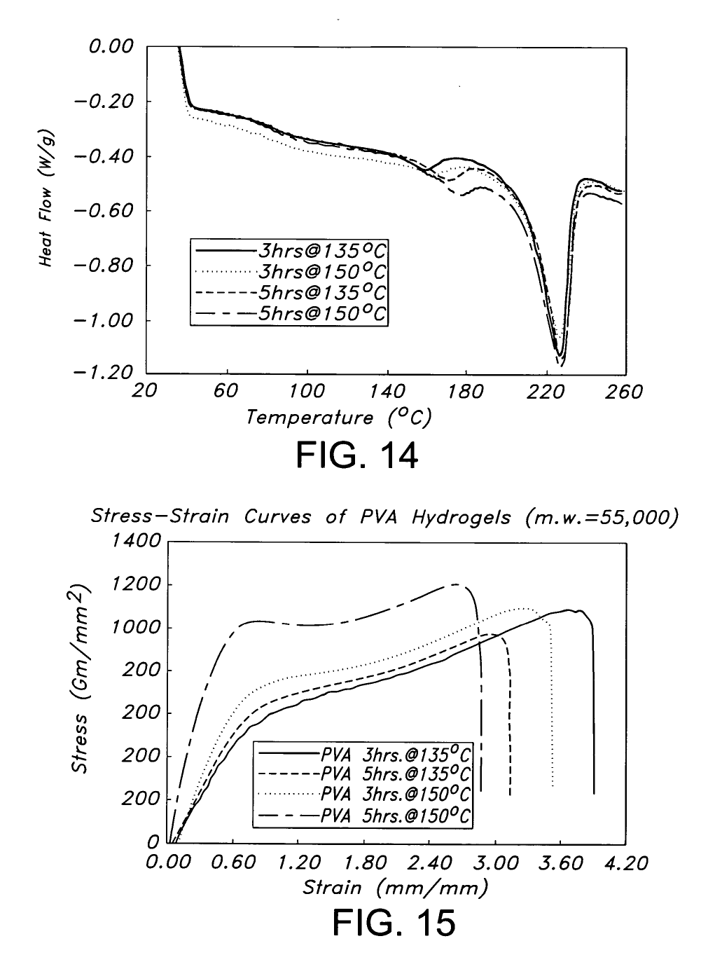

Crystallinity demonstrated direct impact on degree of hydration, mechanical integrity and barrier efficiency. Water dissociated weaker bonds and physical entanglements. DSC thermograms showed that higher transition temperatures and greater areas under curve were observed in films subjected to higher temperature and longer heat treatment cycle. In contrast, dry film did not have such a dramatic differentiation. The thermal shoulder at 165° C., which was observed in dry film, shifted to higher temperature with prolonged heat treatment. This suggests that the thermal process densified the molecular structure and increased the transition temperature assigned to the interfacial proximity between the amorphous and the crystalline phase (FIGS. 13 and 14).

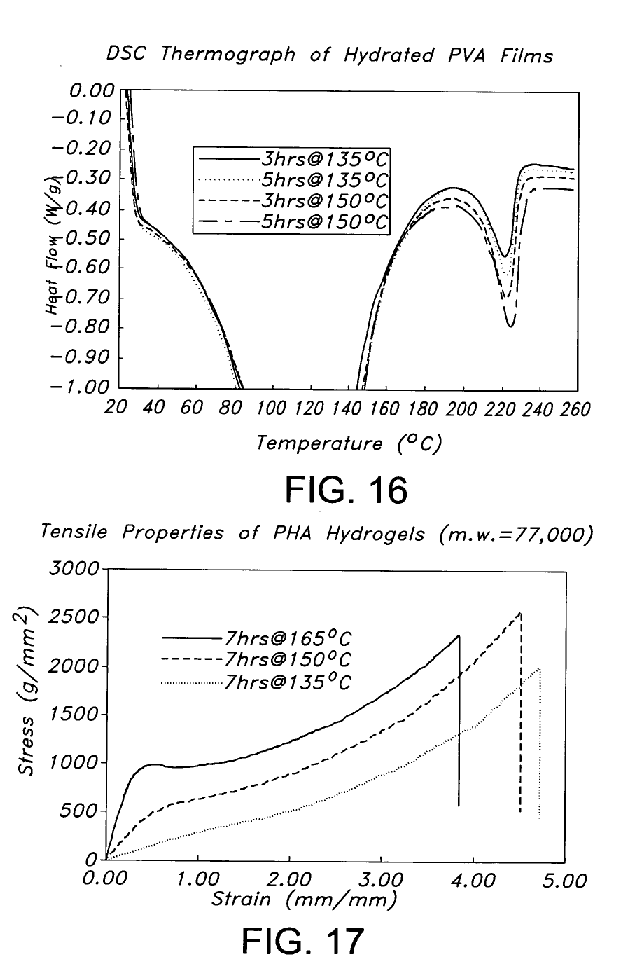

Mechanical characterization generated valuable information on tensile strength, elastic modulus and elongation. With increasing thermal treatment time and temperature, the tensile strength increased. More importantly, the elastic modulus increased proportionally to the prolonged thermal treatment conditions. Elongation of poly(vinyl alcohol) hydrogels decreased as the materials were subjected to higher temperature and time treatment. Elastic modulus were theoretically proportional to the amount of strand density in the specimens. Total strand density was the sum of entanglement density, physical networks and chemical cross-links. The higher the strand density, the smaller the intermolecular weight, since the distance between two points was much shorter. Combining the positive increasing in elastic modulus and the increasing in thermal enthalpy, this suggests that the formation of crystalline phase reinforced the tensile properties and resulted in higher tenacity at the expense of percentage elongation (FIGS. 15 and 16).

Tensile strength and elastic modulus were classic properties to evaluate the thermal process impact on poly(vinyl alcohol) hydrogels. It was observed that prolonged heat treatment led to higher modulus and greater toughness. Multicycle recovery test was designed to test the resilience of bond strength within polymeric materials. PVA hydrogels were subjected to a specified tensile loading under a given cross-head speed, then unloading the force toward to the opposite compressive direction until it reached a non-zero value close to the origin. This procedure was repeated many times. Once the data was collected, permanent deformation and the percentage recovery of each specimen were calculated. Recovery properties can be useful in evaluating the structure-property relationship inherent to the polymeric matrices (Table 2).

TABLE 2

| Process | | | Elonga- | Tough- | | |
|---|---|---|---|---|---|---|
| (m.w. = | Tenacity | Modulus | tion | ness | Recovery | PermSet |
| 77,000) | (g/mm²) | (g/mm²) | (%) | (g/mm³) | (%) | (%) |
| 7 hrs @ | 2137 | 312 | 312 | 3843 | 91.9 | 8.1 |
| 135° C. | | | | | | |
| 7 hrs @ | 2215 | 1004 | 426 | 4416 | 89.2 | 10.8 |
| 150° C. | | | | | | |
| 7 hrs @ | 2058 | 3396 | 360 | 4594 | 86.7 | 13.3 |
| 165° C. | | | | | | |

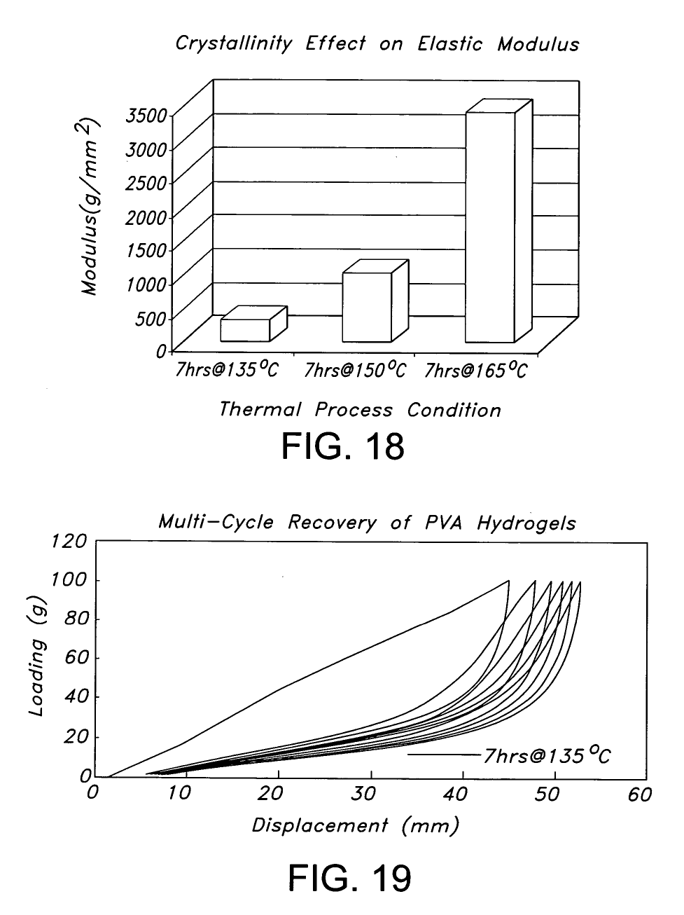

The results showed that higher percentage of recovery was observed in the lightly treated specimens, i.e., hydrogels that were treated at lower temperature. In general, physical networks such as chain entanglements and crystallinity would not be expected to lead to the increase in the degradation temperature for the polymers, whereas the same polymers with chemically cross-linked networks would generally exhibit higher degradation temperature. The second example, during repeated tensile-compressive cycles, rubbers which generally exhibited the typical chemical cross-linked networks would retrieve itself back to near the origin under multiple cyclic tests. This was known as rubber elasticity. In contrast, physical networked polymer such as polyurethane containing soft segments and hard segments linked by either hydrogen bonds or ionic bonds would show incremental permanent deformation over the course of stretching and relaxing. Recovery results showed that poly(vinyl alcohol) hydrogels exhibited the typical performance of physical-networked polymers. This suggests that the enhanced mechanical strength, the reduction in water up-take and the increased melting enthalpy were caused by the crystalline formation promoted by the prolonged thermal process (FIGS. 17 and 18).

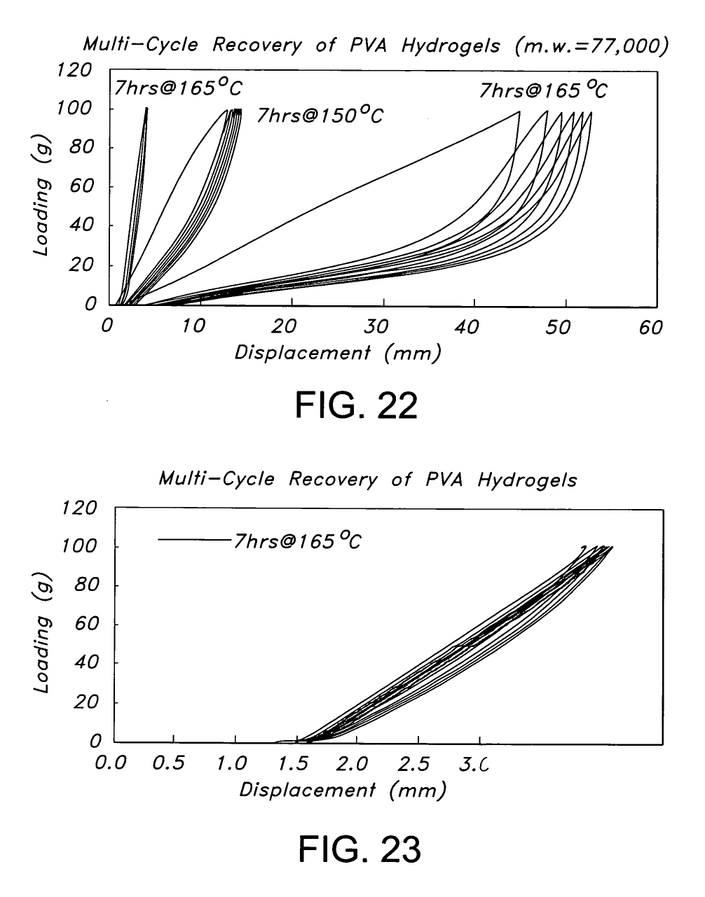

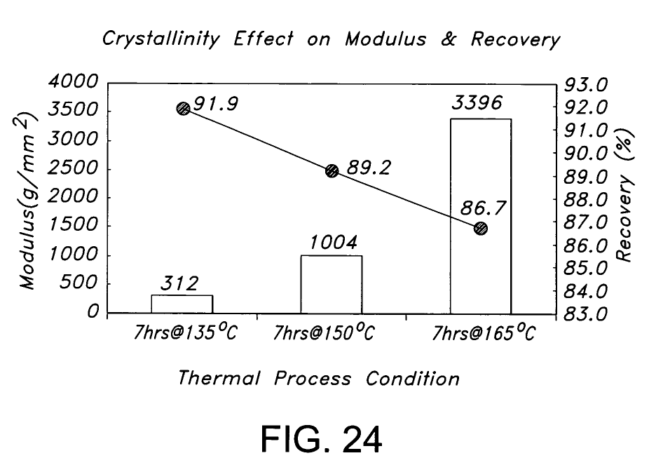

Separate sets of Poly(vinyl alcohol) hydrogels were prepared by using higher molecular weight PVA. Solution preparation and film cast procedures were held to strictly similar requirements. The tensile mechanical results showed an interesting phenomena that under the same thermal treatment condition, higher elastic modulus was observed in the hydrogels made of lower molecular weight, whereas higher tensile elongation was observed in the hydrogels made of higher molecular weight PVA. If there was no crystallinity involved, hydrogels made of higher molecular weight shall have higher elastic modulus based on higher entanglement density generally observed in higher molecular weight polymers. However, when semi-crystalline polymer was selected as the candidate, crystallization process dictated the final mechanical performance of the hydrogels. Although not wishing to be bound by a particular theory, the inventors believe higher molecular weight polymer may experience slower crystallization process because of its large molecules, whereas lower molecular weight polymer may be able to crystallize easily and ultimately reach a higher degree of crystallinity under the same conditions. The larger melting enthalpy, lower water content, higher elastic modulus and the lower elongation of PVA hydrogels made of low molecular weight PVA further confirmed that the formation of crystallinity played a vital role in determining the diffusion properties and mechanical integrity for the medical implants and pharmaceutical applications (FIGS. 19-24).

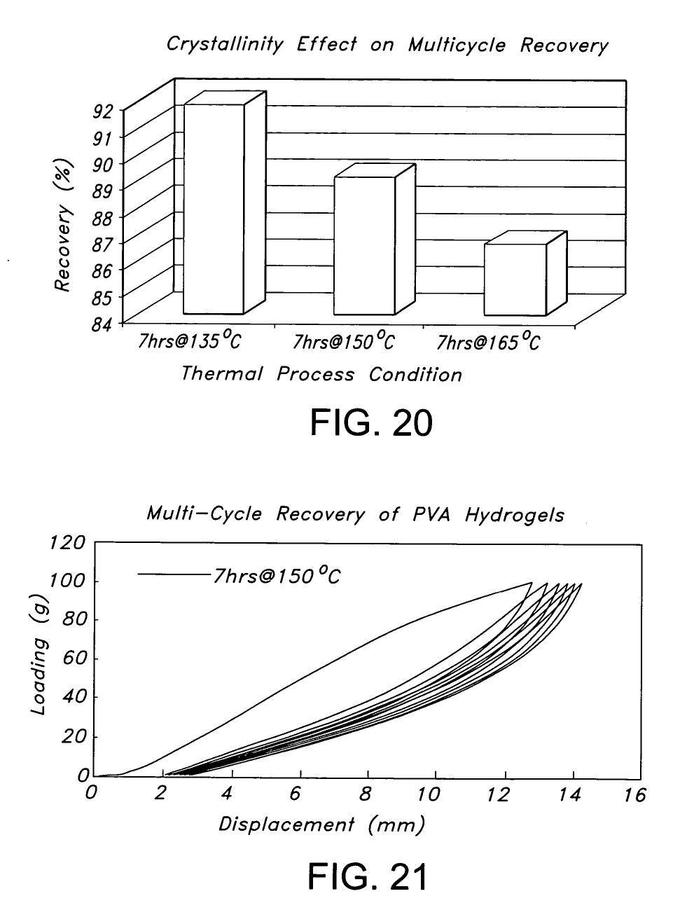

Work has shown that a longer relaxation time was detected in PVA hydrogels subjected to prolonged thermal treatment. Longer relaxation time could be the results of stiffer chain segment and higher strand density. The existence of crystallinity enhanced mechanical strength. It stiffened hydrated PVA films and made them tougher and stronger at the expense of film extensibility (i.e., % elongation). Higher crystallinity increased strand density. The highest modulus, 1643 g/mm², was observed in hydrated PVA films that had been processed in 150° C. for 5 hours. In contrast the lowest tensile modulus, 584 g/mm², was observed in PVA film processed in 135° C. for 3 hours. The crystalline region remained intact even after PVA film was hydrated for three years. Overall structure-property relation suggested that higher process temperature and prolonged heating time had strong impact on water content. Ultimately, the degree of crystallinity would determine the efficiency of the barrier property. Compared to PVA hydrogels made of higher molecular weight PVA (m.w.=77,000), PVA hydrogels made of lower molecular weight PVA (m.w.=55,000) exhibited higher modulus under the same process condition. By alternating the thermal treatment process, hydrogels made of a same grade of PVA(m.w.=77,000) showed distinctive stress-strain curves. As we noticed in FIG. 17, the modulus increased from 312 g/mm² for those processed at 135° C./7 hrs, 1004 g/mm² for those processed at 150° C./7 hrs, to 3396 g/mm² for those processed at 165° C./7 hrs. Moreover, their percent recovery decreased from 91.9%, 89.2% to 86.7%, respectively as shown in FIG. 20.

Crystallinity was sensitive to process condition. Higher melting temperature and larger endotherm enthalpy were consistently associated with prolonged heating condition. Prolonged heat treatment generated higher degree of crystallinity that was verified by lower water content, since water penetrated mostly the amorphous region. PVA film processed in the lower thermal treatment condition, such as 135° C. for 3 hours showed higher water content and lower crystallinity. Mechanical properties further showed that higher crystallinity led to increased modulus in the expense of elongation. By comparing ΔH of dry and hydrated films, the differences in crystallinity were understood. In dry film, the melting enthalpy may be attributed to the combination of crystalline phase and physical entanglement. Heating process promoted higher degree of crystallinity in hydrogels. The difference in enthalpy, ΔH, also suggests that lightly heat-treated PVA film exhibited higher ratio of the amorphous phase, whereas prolonged heating process led to higher ratio of the crystalline phase.

It was interesting to note that hydrogels made of higher molecular weight PVA was associated with lower crystallinity and higher water content. Given all other variables constant, higher entanglement density due to high MW PVA may impede crystalline growth. Thus, a lower degree of crystallinity was observed. Higher degree of crystallinity enhanced mechanical strength by the increase in strand density. Higher strand density led to higher tensile modulus, better mechanical integrity and more reliable dimensional stability. In addition, stress-strain curve showed a significant hump in which only crystallinity polymer exhibited such a feature. Optimizing the concentration of PVA solution and sticking with a purified PVA improved the PVA films and hydrogels. Then, mechanical integrity and barrier efficiency can be achieved in a controlled fashion by controlling the degree of crystallinity. PVA hydrogels with unique semi-crystalline structures allowed us to customize their physical-chemical properties to fulfill the design requirements that were demanded by the pharmaceutical and medical industry.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What is claimed:

1. A method for making a drug delivery device, comprising:
providing a holder made of a material impermeable to passage of an active agent, said holder being shaped to receive a drug core that includes the active agent;
inserting a preformed disc of material having a predetermined degree of crystallinity in the holder; and
inserting the drug core into the holder.

2. The method of claim 1 further comprising the steps of adding a curable liquid to the holder, and inserting a pin in the holder to displace the curable liquid;
curing the liquid and removing the pin;

3. The method of claim 2, wherein the drug core is shaped similarly as the pin.

4. The method of claim 1, wherein the holder includes at least one opening for passage of the pharmaceutically agent.

5. The method of claim 1, further comprising, after insertion of the drug core, covering the drug core with a layer of material.

6. The method of claim 5, wherein the layer of material includes a suture tab.

7. The method of claim 1, wherein the impermeable material comprises silicone.

8. The device of claim 1, wherein the drug core comprises a mixture of the active agent and a matrix material permeable to said active agent.

9. The method of claim 8, wherein the matrix material comprises polyvinyl alcohol.

10. The method of claim 2, wherein the curable liquid comprises polyvinyl alcohol.

11. The method of claim 2, wherein the liquid is cured by heating the device.

12. The method of claim 1, wherein the holder comprises a cylinder that surrounds the inserted drug core.

13. The method of claim 12, wherein an end of the cylinder includes at least one opening.

14. The method of claim 1, wherein the drug core is cylindrical.

15. The method of claim 1, wherein the drug core is coated with a material permeable to said active agent.

16. The method of claim 1, wherein the degree of crystallinity determines the release profile of the device.

17. The method of claim 16, wherein the holder is cylindrical and the disc is circular.

18. The method of claim 17, wherein an end of the cylinder includes at least one opening, and the disc covers the at least one opening.

19. The method of claim 16, wherein the disc is made of a material permeable to the active agent, the permeability being determined by the degree of crystallinity of the disc material.

20. The method of claim 16, wherein the disc is made of crystalline polyvinyl alcohol.

21. The method of claim 16, further comprising, after insertion of the drug core, covering the drug core with a layer of material including a suture tab.

22. A drug delivery device comprising:
a holder made of a material impermeable to passage of a pharmaceutically active agent, and including at least one opening for passage of the active agent therethrough;
a drug core contained in the holder, and including a pharmaceutically active agent; and
a preformed disc made of a material having a known degree of crystallinity and permeable to passage of the active agent, the disc contained in the holder and disposed between the drug core and the at least one opening in the holder.

23. The device of claim 22, further comprising a suture tab attached to the holder.
